(19) United States
(12) Patent Application Publication
(10) Pub. No.: US 2007/0256688 A1
(43) Pub. Date: Nov. 8, 2007

(54) MECHANICAL SINGLE DOSE INTRAPULMONARY DRUG DELIVERY DEVICES

(75) Inventors: Jeffrey A. Schuster, Oakland, CA (US); Peter Holst, Hayward, CA (US); Brian Ament, Hayward, CA (US); Keith Bogdon, Hayward, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: Aradigm Corporation

(21) Appl. No.: 11/737,655

(22) Filed: Apr. 19, 2007

Related U.S. Application Data
(60) Provisional application No. 60/745,334, filed on Apr. 21, 2006.

(51) Int. Cl. A61M 11/00

(52) U.S. Cl. 128/200.23; 424/45

(57) ABSTRACT

Devices for delivering an aerosolized drug formulation and methods for using such devices are herein provided. Specifically, the invention relates to a drug delivery device that contains a drug formulation and an actuator for aerosolizing the formulation in preparation for drug delivery. The drug delivery devices of the invention are configured for delivering a single dose of an active agent (e.g., a pharmaceutical compound) or a mixture of multiple active agents and may further be configured so as to be hand-held, self-contained, portable and disposable. Methods of treatment and drugs that are suitable for use in the subject devices are also disclosed.

### FIELD OF THE INVENTION

The present invention relates to intrapulmonary drug delivery devices and methods of treatment.

### BACKGROUND OF THE INVENTION

Inhalation devices for delivery of therapeutic substances to the respiratory tract of a user are now in common use. Devices for delivering drug formulations to the respiratory tract include metered-dose inhalers, dry powder inhalers and nebulizers. Single dose, disposable dispensing devices that are capable of aerosolizing a formulation for intrapulmonary delivery to a subject are known. One such single dose inhaler, the Alexza Stacatto device, employs chemical heating as an energy source for the delivery of an active agent. The chemical heating unit is built into the device. The chemical heating unit contains heat generating combustion chemicals that interact with a pure active agent (e.g., a drug) that is adhered to the surface of the container. The chemical heating unit is triggered by a percussive or battery operated electronic mechanism which generates the energy necessary for drug vaporization prior to delivery.

There are, however, several problems with the use of a chemical heating system in conjunction with the delivery of active agents. First of all, many active agents, such as nucleic acids and proteins, are very delicate and are easily denatured under extreme environmental conditions, for instance, high temperatures. The use of a heating system in conjunction with the delivery of delicate active agents can cause the molecules of the drug to be denatured thereby rendering them ineffective for their desired function. Additionally, because the active ingredient in this system must be stored in contact with the metal substrate, denaturation of the drug can occur on storage, rendering the drug ineffective or even toxic. Other problems include the fear of burning, release of noxious emissions, explosion and other hazards that are associated with the use of chemical heat actuated inhalers.

Many drugs are formulated so as to be contained and delivered within a delivery enhancement agent such as a liposome, micelle, polymer, dendrimer, nanotube, buckyball, micro or nanoporous structure, a colloidal system or the like. The use of a heating system in conjunction with the delivery of an active agent that is encapsulated within such a delivery enhancement agent can cause the breakdown of the delivery enhancement agent and thereby prevent the effective, controlled release and delivery of the drug.

Single dose, disposable dispensing devices that do not employ a chemical heating system in conjunction with the delivery of an active agent have also been proposed. One such technology involves the use of a force generated by the subject's own inhalation to aerosolize the active agent. The active agent contained in such devices are formulated as dry particulate matter that is engineered for high dispersibility. However, the amount of energy that a person can deliver by an inhalation is severely limited, and there are several medical conditions, e.g. asthma, COPD, emphysema, and the like, that may further reduce a patient's ability to generate a sufficient inhalation force so as to cause the accurate and precise aerosolization and delivery of the drug. Additionally, such devices are not compatible for use with liquid formulations and require each dry powder drug formulation to be individually formulated for dispersibility, which leads to increased development times, costs and higher risks. Another drawback is that high dispersibility runs counter to the control of the powder required for high volume packaging of the powder.

Thus, there remains a need, which has not been adequately met by the prior art, for a single-use inhaler that is suitable for dispensing an aerosolized formulation-based drug without the need for heating units, which is compatible with the delivery of liquid formulations, and does not require the patient to provide the inspiratory force to aerosolize dry powder and/or liquid drug formulations. The present invention addresses these needs.

### SUMMARY OF THE INVENTION

Devices for delivering an aerosolized drug formulation and methods for using such devices are herein provided. Specifically, the invention relates to a drug delivery device that includes a drug formulation and an actuator for aerosolizing the formulation for drug delivery. The drug delivery devices of the invention may be configured for delivering a single dose of an active agent (e.g., a pharmaceutical compound) or a mixture of multiple active agents and preferably are configured as hand-held, self-contained, portable and disposable devices. Methods of treatment and drugs that are suitable for use in the invention are also disclosed.

The invention can include a single dose drug delivery device or a system which is comprised of a plurality of single dose drug delivery devices. The device includes a source of stored energy, a trigger mechanism for releasing the stored energy and a container which forms a part of the device and is integral with the device. The container holds a flowable formulation which consists of only a single dose of a pharmaceutically active drug. The device includes a mechanism for transferring the stored energy to the container so as to force the formulation out of the container. The formulation may form an aerosol which can be inhaled by a patient. The patient can be treated by using the device and then disposing of the device and using a new device. The system can be set up as a plurality of devices which provide a certain regimen of treating whereby the patient uses one of the devices (out of 2, 5, 7, 10, 20, 21, 28, 30 or more), disposes of it and at the next treatment uses a completely new device. The devices may be individually packaged and/or packaged as a group. Further, when packaged as a group the devices may be labeled for a particular date and/or time where the device is to be used.

In certain embodiments, a suitable dispensing device of the invention includes an actuator that is configured for aerosolizing a contained formulation. The actuator includes an energy source, such as a compressed gas or a mechanical spring, which is configured for storing and transferring energy to a contained formulation in a manner sufficient to aerosolize the formulation. A suitable formulation is a flowable liquid or dry powder formulation, which includes an active agent or a combination of active agents to be delivered, for instance, one or more pharmaceutically active drugs. A single dose of the formulation to be delivered is packaged in a container which loaded into the dispensing device to form a system that can be used in a method of delivering drugs to a subject, for example via the ocular, nasal or intrapulmonary route, for topical or systemic effect, or both. In the preferred embodiment, the dispensing device is a single use, disposable device which is capable of aerosolizing substantially all the contents of the container for the controlled delivery of liquid or dry powder drug formulations to a subject, by the pulmonary route.

The actuator of the invention confers several advantages, among which is that it provides a simple, compact, low cost, and effective means for aerosolizing a contained formulation. The actuator additionally includes novel safety features such as a very low gas charge, a locking mechanism or latch that prevents accidental actuation during storage and transport, and additional safety mechanisms, for instance, tear-off bands or removable blocks, which provide additional protection against accidental triggering such that the device can be handled and manipulated in a way that readies the device for use without triggering an actuation. The devices of the invention have many other benefits including that they are small, lightweight, low cost and safe to use.

These and other objects, advantages, and features of the invention will become apparent to those persons skilled in the art upon reading the details of the invention, as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIGS. 1A and 1B. FIG. 1A is a general external view of the first embodiment of the drug delivery device, in this embodiment an inhaler; FIG. 1B is a cross-sectional view of the inhaler of FIG. 1A at the same viewing angle.

FIG. 2 is a longitudinal sectional view of the inhaler, fully loaded with drug, as would be supplied in disposable form.

FIG. 3 is a plot of Weber number versus particle diameter.

FIG. 4 is a plot of kinetic, surface, and thermal energy of a water droplet versus diameter.

FIGS. 5A and 5B. FIG. 5A shows a longitudinal sectional view of the gas actuator of the first embodiment of the inhaler before use with the latch in its safety position; FIG. 5B shows on a larger scale the latch used in FIG. 5A.

FIGS. 6A, 6B, and 6C show diagrammatically part of the embodiment of FIGS. 5A and 5B, in three successive stages, namely with the latch in its safety position, with the latch in its non-safety position prior to firing, and with the latch in its position during firing.

FIGS. 7A and 7B. FIG. 7A is a longitudinal sectional view of the actuator as would be supplied with a mechanical spring. FIG. 7B shows the right-hand portion of the actuator of FIG. 7A, on a larger scale.

FIG. 8 is a view corresponding to FIG. 7A, but showing the nut rotated in a first direction to create an impact gap between the piston face and the piston.

FIG. 9 shows the actuator with the nut screwed out to set the stroke of the piston.

FIG. 10 corresponds to the previous views of the actuator with a mechanical spring, but showing the components in a position immediately after actuation, with the sliding sleeve disengaging the latch.

FIGS. 11A and 11B. FIG. 11A is an enlarged longitudinal sectional view of the latch; FIG. 11B is an enlarged end view of the latch;

FIG. 12 is an AERx strip used in the embodiments for the delivery of liquid drug formulations.

FIG. 13 is a table of emitted dose data using an inhaler of the invention with 0.6 micrometer nozzles and sodium cromoglycate at actuator gas masses of 35 mg and 40 mg.

FIG. 14 contains particle size distribution data using an inhaler of the invention with 0.6 micrometer nozzles and sodium cromoglycate at actuator gas masses of 35 mg and 40 mg.

FIG. 15 is a table of emitted dose data using an inhaler of the invention with 0.4 micrometer nozzles and sodium cromoglycate at actuator gas masses of 40 and 45 mg.

FIG. 16 contains particle size distribution data using an inhaler of the invention with 0.4 micrometer nozzles and sodium cromoglycate at actuator gas masses of 45 mg, 40 mg and 35 mg.

### DETAILED DESCRIPTION OF THE INVENTION

Before the present device and method embodiments of the invention are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a channel” includes a plurality of such channels and reference to “the element” includes reference to one or more elements and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The terms “aerosol” and “aerosolized formulation,” and the like, are used interchangeably herein to refer to a volume of air which has suspended within it particles of a formulation comprising a drug or diagnostic agent wherein the particles have a diameter in the range of 0.5 to 12 microns, for respiratory therapy, or in the range of 15 to 50 microns for ocular therapy, or in the range of 2 to 30 microns, preferably 10 to 20 microns, for nasal delivery.

The term “airway” refers to the part of a device of the invention, such as an inhaler, where gas flows, and includes the region where the aerosolized drug becomes entrained into the inspiratory flow of the patient.

The term “nozzle”, “pore” and the like will be used hereafter to describe a hole in a sheet through which a liquid formulation is forced to form an aerosol. A pore can be created using a wide variety of techniques, including but not limited to laser micromachinging, MEMs, casting, molding, electric discharge, embossing, track etching, and the like. In the preferred embodiment, pores are UV laser micromachined whereby each pore has a sub-micrometer diameter. Nozzles have diameters that are preferably in the range of about 0.2-20 micrometers, more preferably in the range of about 0.3-1.2 micrometer, most preferably ranging from about 0.4 micrometers to about 0.6 micrometers.

The term “array” or “nozzle array” or “pore array” and the like will be used hereinafter to describe a grouping, collection and/or plurality of pores in a sheet that could be arranged in a variety of geometric configurations. This collection of pores can be described geometrically in either rectangular or circular coordinates or any other coordinate system, or be randomly distributed. For example, an array could be a plurality of pores arranged in as a rectangular array with rows and columns or a radially configured array. The array could contain a continuous or discrete gradient (variation) in the size of the pores. Various embodiments of the invention have been described hereinafter.

The term “chemical emitted dose”, “emitted dose” and “delivered dose”, as used herein, refers to an amount of pharmaceutically active drug or diagnostic agent delivered to a patient from an aerosol generated from a formulation. The said aerosol will exhibit a characteristic particle size distribution that is the result of the liquid formulation and the porous sheet, among other factors.

The terms “container”, “package” and the like are used herein to refer to a receptacle for holding and/or storing a drug formulation. The container can be single-dose or multidose, and/or disposable or refillable. In the preferred embodiment, it contains a single dose of the active ingredient, and is a single use disposable rather than refillable. The container also may have features that ensure the stability of the formulation, ensure sterility, limit water egress/ingress, and facilitate the presentation of the formulation for aerosolization.

The term “damping medium” refers to a fluid that is used to attenuate the impact of the actuator against the container such that the force does not rupture the container, and other optional features such as pores of a delivery strip. The damping medium may be contained between a piston and a sleeve, is preferably comprised of but not limited to a silica thickened, viscous, synthetic hydrocarbon lubricant grease. Many other damping mediums may be used, including air and other gasses, various varieties of oil and grease, and any of a wide variety of viscous media.

The term “delivery strip” shall mean any single-use disposable dosage form used for the storage of the formulation and also containing features that aid in the generation of the aerosol, such as pores that act as nozzles. In a preferred embodiment, this dosage form is a laminate: a blister is drawn into the bottom layer, the middle layer is a vapor barrier, and the top layer is a polymer sheet containing a nozzle. This container/nozzle system is filled with liquid formulation under clean room conditions and disposed of after the container contents are dispensed. The single use feature removes any possibility of the clogging that can limit the lifetime and repeatability of multi-use nozzle systems. An example of a suitable “delivery strip” is an described in U.S. Pat. No. 5,497,763, incorporated herein in its entirety by reference.

The term “emitted dose,” as used herein, refers to the amount of aerosolized drug emitted from a drug delivery device such as an inhaler that is available for a patient. Emitted dose is frequently abbreviated ED and expressed as a percentage of the dose packaged in the device.

The terms “formulation” and “flowable formulation” and the like are used interchangeably herein to refer to any pharmaceutically active drug or combination of drugs, in conjunction with any suitable excipients, that are contained within the device of the invention. In a preferred embodiment, the drug a respiratory drug, or drug that acts systemically, or a diagnostic agent that is suitable for respiratory delivery. The formulation will have properties such that it can be aerosolized by the device, for example into an aerosol comprising particles having a diameter of 0.5 to 12.0 microns for respiratory therapy, or 15 to 75 microns for ocular therapy. Such formulations may be dry, such as dry powders, but are preferably liquids, including but not limited to aqueous solutions, ethanolic solutions, aqueous/ethanolic solutions, colloidal suspensions and microcrystalline suspensions. Preferred formulations are drug(s) and/or diagnostic agent(s) dissolved in a liquid, preferably in water. Preferred excipients include tonicity adjusters such as salts, bacteriocidal or bacterostatic agents, surfactants such as polysorbate 20 or polysorbate 80, and other pharmaceutically acceptable ingredients.

The term “gas mass,” as used herein, refers to the amount of gas that is filled into the energy storage means of the inhaler. Higher gas masses generate higher pressures and flow rates in the actuator that correspondingly increase the rate of aerosolization and the efficiency of the dispersion of the drug particles in the invention.

The term “gradient”, as used herein, refers to a variation in the individual pore size within the plurality of pores formed on the sheet. This gradient could take on the form of being either a continuous or discrete change in the pore size within the plurality of pores. The gradient can also have the characteristic of being a negative or positive gradient; wherein, the negative gradient represents a decreasing pore size with respect to the direction of the airflow across the porous sheet or in the case of a positive gradient having an increasing pore size with respect to the direction of the airflow across the porous sheet. Although it is preferred that the gradient be a linear gradient, i.e. a continuous change across the sheet, any gradient may be used, including a discrete change, a parabolic profile, or any other profile.

The terms “individual”, “subject”, or “patient”, used interchangeably herein, refers to an animal, preferably a mammal, generally a human; that is the delivery target of the invention.

The term “actuator” and the like is used to describe a system that includes an energy store for the source of energy required to form an aerosol, a trigger, switch or other component for releasing said stored energy upon an action of the subject, such as pressing a button or inhaling from the device, and a piston, ram, gas pressure or other component for delivering said energy to the formulation and/or a device for forming the aerosol. In a preferred embodiment, the actuator is one that has been previously disclosed as the energy source for a needle free injector, the “intraject actuator,” described in U.S. Pat. No. 6,620,135, included herein in its entirety by reference. The energy source of the invention can be any form of stored or potential energy, such as but not limited to a pressurized gas, spring of any type stored chemical components that release energy in a controlled chemical reaction, or a mechanical metal coiled spring. When using a mechanical spring, the tension on it may be varied in order to tune the aerosolization of various drug formulations of various viscosities and other properties. When using pressurized gas, the aerosolization of various drug formulations may also be tuned by varying the gas mass contained in the actuator.

The term “jet” is used herein to describe the column of liquid that exits a pore as the liquid formulation is forced through a porous sheet under pressure.

The term “MEMs,” as used herein, refers to a micro-electro-mechanical-system that can be used for making nozzle arrays having micron-scale pores.

The term “ooze” is used herein to describe liquid formulation that exits the pore as a relatively large droplet or droplets of non-aerosolized liquid as opposed to being a jet or an aerosol, mist or spray.

The term “porosity” is used herein to refer to a percentage of an area of a surface area that is composed of open space, e.g., a pore, hole, channel or other opening, in a film, sheet, nozzle, filter or other material. The percent porosity is thus defined as the total area of open space divided by the area of the material, expressed as a percentage (multiplied by 100). High porosity (e.g., a porosity greater than 50%) is associated with high flow rates per unit area and low flow resistance. In general, the porosity of the nozzle is less than 10%, and can vary from 10.sup.−3% to 10%, while the porosity of the filter is at least 1%, and preferably it is at least 50% porous.

The terms “particle size distribution,” “size distribution”, “aerosol size distribution” and the like as used herein refer to the distribution of particle sizes of an aerosolized drug emitted from a device of the invention, such as an inhaler, that are available for a patient to inhale. Particle size distribution is frequently abbreviated PSD and can be expressed by the percentage of an ED per micrometer at each of several diameters. In order to simply the presentation of PSD data, they are often reduced to a presentation of the overall mass median aerodynamic diameters MMAD, defined as the aerodynamic diameter at which half of the drug is in larger particles, and half is in smaller particles. Aerodynamic diameter is the physical diameter of a particle of density 1 gm/cc that would settle at the same rate as the actual particle. PSD determinations are often made by light scattering techniques phase doppler anemometry, or cascade impaction. Data may presented as percentiles, denoted as ×N, meaning the amount of the aerosol in particles smaller than diameter N. The width of the distribution can be presented as ratios of percentiles, such as ×84/×50, or ×50/×16. For aerosols that have a log normal distribution, ×84/×50 and ×50/×16 are the geometric standard deviation (GSD).

The terms “porous sheet” and “porous film”, used interchangeably herein, refer to a sheet of material having any given outer parameter shape, but preferably having a convex shape, wherein the sheet has a plurality of pores therein, which openings may be placed in a regular or irregular pattern, and which pores have an unflexed diameter of their exit aperture in the range of 0.25 micron to 50 microns and a pore density in the range of 1 to 1,000 pores per square millimeter. The porous sheet has a porosity of about 0.0005% to 0.2%, preferably about 0.01% to 0.1%. In one embodiment, the porous sheet comprises a single row of pores on, e.g., a large piece of sheet material. The pores may be planar with respect to the surface of the porous sheet material, or may have a conical configuration. The sheet may be a polymer film, a metal, a glass, a ceramic or any other pharmaceutically suitable engineering material.

The term “sodium cromoglycate,” as used herein, refers to a drug, also known as cromolyn sodium, that is used in the treatment of asthma. This drug is herein used as a reference standard when comparing the data generated from new inhalers against existing technologies.

The term “Weber number,” as used herein refers to a number that is useful in analyzing fluid flows, including where an interface exists between 2 different fluids (i.e. multiphase flows), thin film flows, and the formation of droplets and particles. In the delivery of pharmaceutical drug formulations via the pulmonary route, the Weber number is helpful in understanding the fluid dynamics required to aerosolize drugs. In general, when the Weber number is less than 1, surface effects dominate the energetics, whereas when the Weber number is greater than 1, kinetic energy dominates.

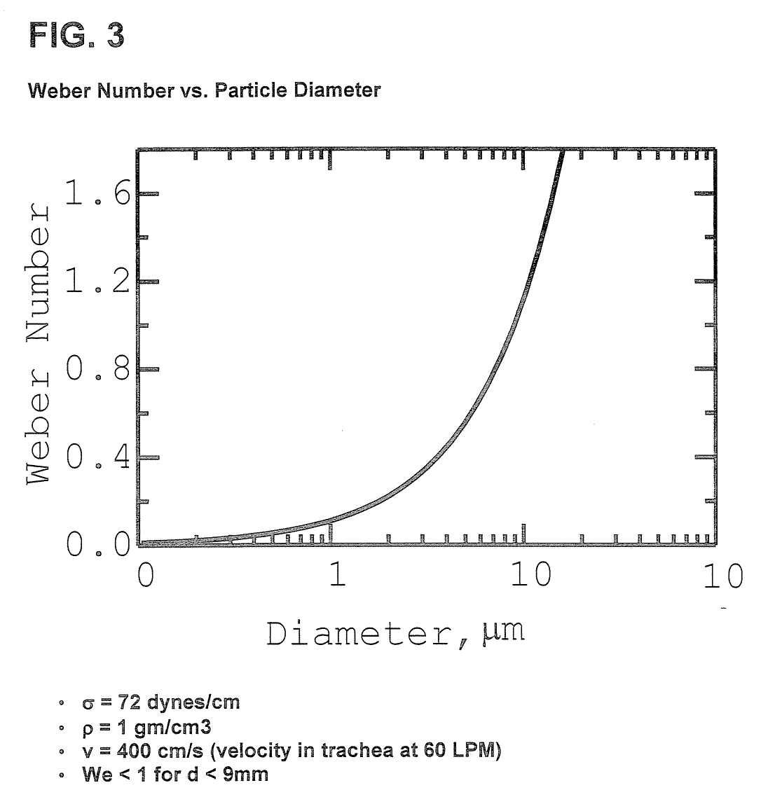

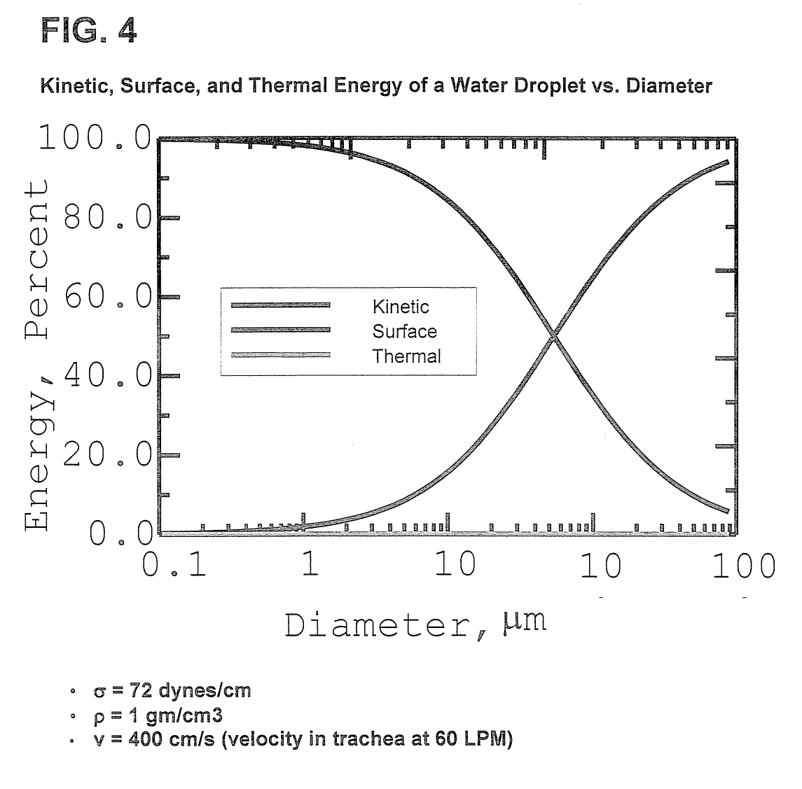

Understanding the science behind creating aerosols from liquid and dry powder formulations for pulmonary delivery requires an understanding of FIG. 3, a plot of Weber number versus particle diameter, and FIG. 4 that is a plot of the kinetic, surface, and thermal energy of a water droplet versus diameter. The Weber number, referenced in FIG. 3, may be written as:
We=ρv²d/σ
where:
ρ is the density of the fluid
v is its velocity
d is its characteristic diameter
σ is the surface tension.

The Weber number is useful in analyzing fluid flows, including where an interface exists between 2 different fluids (i.e. multiphase flows), thin film flows, and the formation of droplets and particles. In the delivery of pharmaceutical drug formulations via the pulmonary route, the Weber number is helpful in understanding the fluid dynamics required to aerosolize drugs. This is important because the desired mass median aerodynamic diameter (MMAD) is generally desired to be in the range of about 0.5-5 micrometers, preferably about 1.0 to 4.0, more preferably about 1-3 micrometers for the systemic delivery of pharmaceutical drug formulations via the pulmonary route. The equation above indicates that the Weber number is inversely proportional to the surface tension of the fluid, and directly proportional to the density of the fluid, the size of the fluid particles, and the square of the velocity of the particles. Thus, a low Weber number means that more energy is required to aerosolize a drug formulation that may be in the liquid or solid phase by forcing another phase, such as air, across the first phase. In general, when the Weber number is less than 1, surface effects dominate the energetics, whereas when the Weber number is greater than 1, kinetic energy dominates.

As the particle diameter decreases into the size range that is important for pulmonary drug delivery (˜1-4 micrometers), FIG. 3 shows that the Weber number correspondingly decreases below one at approximately 9 micrometers. FIG. 3 assumes a surface tension of 72 dynes/cm, a density of 1 gm/cc, and a velocity of 400 cm/s, numbers that approximately correspond to the inhalation of aqueous droplets. This shows that generation of particles in the respirable range for inhalation occurs in a fundamentally different regime of physics than more familiar aerosol droplets of most applications which are greater than 10 micrometers, implying that novel methods are required. FIG. 3 also shows that relatively more energy will be required to aerosolize a drug into the desired range of smaller particles of 1.0 to 4.0 micrometers in size such that they are entrained into the inspiratory flow of a patient, and choice of energy source will be a key determinant of performance.

The plot of FIG. 4 confirms the analyses of the Weber number and FIG. 3 via an example that is a plot of kinetic, surface, and thermal energy of a water droplet versus diameter. FIG. 4 also shows that as particle diameter decreases into the range that is appropriate for pulmonary delivery, the surface energy becomes the greatest percentage of the energy required for the formation of a droplet of water.

### Representative Components of a Device of the Invention

Devices for delivering an aerosolized drug formulation and methods for using such devices are herein provided. Generally, the invention relates to a drug delivery device that contains a drug formulation and includes an actuator for aerosolizing the formulation in preparation for drug delivery. The aerosolizing device may include one or more of the following: a housing, a container containing a suitable drug to be delivered, an aerosolization mechanism, and an actuator, for instance, a mechanical actuator.

An aerosolizing device of the invention may include a housing. The housing may have any shape, for example, ellipsoidal, rectangular, square, or may take the form of a regular prism, for example, a triangular, rectangular, pentagonal prism, and the like. Additionally, the device need not possess any axial symmetry, as long as fluid flow is directed through an included fluid channel (e.g., substantially all of the fluid flows through the fluid channel). Additionally, it is not necessary for the device to be straight. For example, the device may be curved along an arc. However, it is preferred that the portion of the fluid channel that contains the aerosol to be substantially straight, to minimize the possibility of aerosol impaction in the device.

In certain embodiments, the aerosolizing device is configured as an inhaler, which includes a mouthpiece that is adapted for allowing a subject to inhale a drug to be delivered. The mouthpiece may be detachable and the housing may be configured for storing the mouth piece before or after use. For instance, the housing may include a storage location on said housing whereby said storage location is configured for storing a removable mouthpiece. The housing may also contain a mouthpiece location adapted for attaching the removable mouthpiece in preparation for use of the device.

The housing may be configured for holding and interconnecting a container, which contains a suitable drug formulation, the actuator, and the mouth piece and aerosolization mechanism (if included). A suitable drug formulation to be aerosolized and delivered may be a flowable composition such as an liquid or dry powder formulation of an active agent, such as a pharmaceutical compound, which may be packaged as a single dose. The container may be preloaded into the device during assembly by the manufacturer or may be loaded after manufacture of the housing (e.g., right before use), in which case the housing may contain an orifice and a cavity that is configured for the loading of the container into the housing of the device. The housing is configured for facilitating the actuation and interaction of the actuator with the drug container in a manner sufficient to aerosolize and deliver a single dose of a contained formulation.

The container may be fabricated from any suitable material dependent upon the formulation of the drug to be delivered and the desired functioning of the device. For instance, the container may be rigid, semi-rigid or flexible and may be fabricated from a variety of materials such as thin polymer films, medical grade metals and plastics, glass, ceramics and the like. In certain embodiments, the container is fabricated from a porous material so as to allow the passage of a compressed gas through the container. The container may have one surface or a plurality of surfaces. The proper materials will be chosen for proper drug contact properties, sterility, and to prevent water ingress/egress.

The container includes a lumen which contains a formulation to be delivered to a subject. The container is preferably configured to deliver a single dose, for instance, a single bolus of aerosolized formulation. For instance, the container may be pre-filled with a liquid or dry powder formulation that is to be aerosolized and inhaled by a user of the device. In a preferred embodiment, delivery strips that can be used with the invention include but are not limited to those described in U.S. Pat. No. 5,497,763, U.S. Pat. No. 5,709,202, U.S. Pat. No. 5,718,222, U.S. Pat. No. 5,823,178, U.S. Pat. No. 6,014,969, U.S. Pat. No. 6,070,575, U.S. Pat. No. 6,354,516, and U.S. Pat. No. 6,855,909, incorporated herein in their entirety by reference.

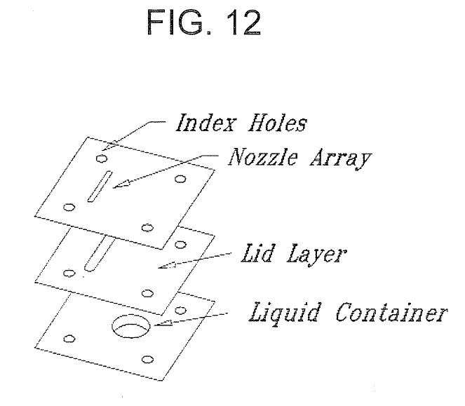

FIG. 12 is a view of a representative drug formulation container for use in certain embodiments of the invention. The container may be a multi layer laminate, that includes a blister drawn into the bottom layer, a middle layer that is configured as a vapor barrier and a top or nozzle layer. The nozzle layer may be specially formed, for instance, as a pore array. Each layer may comprise a single material or be itself a multi-layer laminate, with various materials in the multi-layer laminate being chosen for various properties, including but not limited to drug contact properties, water vapor barrier, mechanical structure, sterility, clarity, ease of manufacture, and the like. In one embodiment, the drug contact surface comprises polyethelene, the nozzle layer comprises polyetherimide, the blister layer comprises polychlorotrifluoroethylene (PCTFE), and the lid layer comprises aluminum.

In certain embodiments, at least one surface of the container or a portion thereof is configured for moving. For instance, the container may be configured for being compressed or may include a surface, for instance, a moveable wall that is configured for moving when a sufficient force is applied to the wall. Additionally, in certain embodiments, the container includes a surface which contains one or more pores, for instance, a plurality of pores. In certain embodiments, a surface of the container is configured as a sheet containing a plurality of pores. In one embodiment the container includes an opening covered by a sheet having a plurality of pores therein.

The nozzle layer of the container may include small pores in a thin sheet. The material used may be any material from which suitable pores can be formed, which has mechanical properties that will withstand the pressures required for aerosolization, and which does not adversely interact with other components of the delivery device, particularly with the formulation being administered. The sheet materials that can be used for forming at least a portion of the container which contains pores include, but are not limited to flexible and non-flexible sheets, that are either organic or inorganically based. An example of a flexible, organic sheet could include materials such as, but not limited to polycarbonates, polyimides, polyamides, polysulfone, polyolefin, polyurethane, polyethers, polyether imides, polyethylene and polyesters. Co-polymers or shape memory polymers can also be used. Examples of non-flexible, inorganic sheet materials can include, but are not limited to, aluminum, gold, platinum, titanium, nickel, alloys of steel, silicon, silica, glasses, and cepistonics. The thickness of the sheet material has effects on both the manufacturing and configuration of pore design as it relates to aerosol performance. The sheet is preferably from 10 to about 200 micrometers in thickness, more preferably from 20 to 100 micrometers, and most preferably about 12 to 45 micrometers in thickness. In the preferred embodiment, the thickness is about 25 micrometers. In one embodiment, the material is a flexible polymeric organic material, for example a polyether, polycarbonate, polyimide, polyether imide, polyethylene or polyester. Flexibility of the material is preferred so that the nozzle can adopt a convex shape and protrude into the airstream upon application of pressure, thus forming the aerosol away from the static boundary layer of air.

Considerations for the membrane material include the ease of manufacture in combination with the formulation container, flexibility of the membrane, and the pressure required to generate an aerosol from pores spanning a membrane of that thickness and flexibility. The pores in the nozzle can be any size and shape that will form aerosols suitable for drug administration, but in certain embodiments optimized for pulmonary administration, have exit diameters that are substantially round and have diameters in the range of about 0.1 to about 20 micrometers, including about 0.2 to about 2 micrometers, or about 0.4 to about 1 micrometer. Depending on the pressure used and the pore diameter, any number of pores can be used, including 1 or two pores.

Methods for generating pores in thin sheets of material are well known in the art, for instance, U.S. Pat. No. 6,732,943 describes methods used to form pores that uniformly penetrate a thin sheet of material. These methods typically utilize the energy of a laser source directed onto the sheet so as to form pores through the sheet. The pores can be formed either individually or in plurality with a single or multiple groupings of arrays of pores on the sheet. The laser source may be controlled using a mask and/or beam-splitting and/or focusing techniques. Alternatively, groups of pores in sheets of material may be formed in a non-uniform manner, for instance, pores or groups of pores may be formed that exhibit deliberate gradation or discrete step changes in the pore sizes contained with the group. The inclusion of a gradient or discrete step change in pore size is accomplished during the formation of the pores in the sheets.

The pores on the sheet may be arranged in rectangular arrays, such as in rows and columns or grids of pores at regular, substantially uniform distances from one another. Alternatively, the pores may also be arranged in a circular fashion or some other geometric orientation where the subsequent rows or rings of pores can be described in radial coordinates. Other geometries could also be used, or the pores could be randomly distributed.

The pores formed on the sheet may be cylindrical or conical in shape. In the example of cylindrical pores, the pores pass perpendicularly through the sheet maintaining approximately the same diameters at the entrance and exit sides of the sheet. In a preferred embodiment, the pores are larger on the side of the sheet to which formulation is applied under pressure, and become smaller in diameter, reaching a minimum diameter on the opposing side of the sheet. This minimizes the pressure required to generate the aerosol. The shape of the pore walls can take on either a straight or curved taper in the case of the conical pores. The pores can also have a stepped configuration, wherein the first section of the pore is a relatively large hole, having in its base a smaller cylindrical, or conical shape, or any combination of conical sections, straight sections, and steps.

The diameter of the pores may vary dependent upon the nature of the formulation to be aerosolized and delivered. The diameter of the pores should be such that a formulation forced through the pores is aerosolized to particles having a diameter of about 0.1 μ to 1000 μ, including about 1 μ to about 100 μ, or about 5 μ to about 50 μ. The diameter of a pore may be about 0.01 μ to about 1000 μ, including about 0.02 μ to about 400 μ, about 1 μ to about 100 μ or about 5 μ to about 50 μ. In one embodiment, two holes are used to create liquid jets that impinge on each other, forming an aerosol smaller in diameter than the jets. In certain embodiments, the number of holes is from about 100 to about 1000, including about 200 to about 600 holes, or about 300 to about 550 holes. The holes can be of any profile, but preferably taper, growing smaller from the entrance to the exit side to minimize the pressure required for the aerosolization. The entrance side may be larger than about 5 micrometers in diameter, and may be greater than about 10 micrometers in diameter or greater than about 15 micrometers in diameter. The attributes of the pores in the surface of the container or porous sheet facilitate the desired control over the particle size distribution (PSD) and emitted dose (ED) of an aerosol to be produced. In certain embodiments the pores are provided in a pore density of about 1×10³ to about 1×10¹⁰ pores/cm² or about 1×10⁵ to about 1×10⁸ pores/cm² and may have a diameter in the range of about 0.4 to about 5 microns.

In one embodiment, the actuator of the device releases a pressurized gas which is in fluid communication with a container that contains a dry powder formulation. The released gas supplies the energy necessary for aerosolization by flowing through the dry powder formulation, overcoming the surface interactions of the powder particles and causing the dry powder particles to be dispersed through the pores in the porous sheet which thereby aerosolizes the dry powder formulation. The aerosol may then be introduced into an optional turbulence chamber to aid in dispersion, and subsequently delivered to a mouthpiece for inhalation by the subject.

In another embodiment, composition in the container is a liquid formulation, and the device includes an aerosolization mechanism, for example a nozzle or an array of nozzles. The container includes a moveable wall which is in communication with the actuator. Once actuated, the actuation mechanism interacts with the container by transferring energy to the movable wall of the container in a manner sufficient to compress the container. An aerosol of the liquid formulation is produced by energizing the liquid composition and thereby causing the liquid formulation to pass through the sheet containing an array of nozzle pores. The volume of enclosed formulation may be about 10 to about 200 microliters, preferably about 25 to about 100 microliters, more preferably about 50 microliters.

Many drug containers could be used with the invention, including but not limited to polymers, glasses, and metals, and a nozzle or nozzle array may or may not be directly incorporated onto the drug container. U.S. Pat. Nos. 5,497,544, 5,544,646, 5,497,763, 5,544,646, 5,718,222, 5,660,166, 5,823,178 and 5,829,435, incorporated herein in their entirety by reference, describe devices and methods useful in the generation of aerosols suitable for drug delivery. These devices generate fine, uniform aerosols by passing a formulation through a nozzle array having micron-scale pores as may be formed, for example, by LASER ablation or MEMs. Any drug container and aerosolization apparatus can be used with the invention that is consistent with requirements for drug stability, shelf life, sterility, and the like.

In certain embodiments, the container includes a turbulence chamber and a channel. For instance, the container itself can be configured to both contain the formulation (e.g., a dry powder formulation) and to perform as a turbulence chamber, or the container may optionally be attached to a separate turbulence chamber that is designed to accept an aerosolized powder formulation via a channel that opens between them such that when the actuator is actuated, the dry powder drug formulation is forced into the optional turbulence chamber and subsequently out of the device and to the patient, thereby dispersing the powder into particles about 0.1 to about 10 micrometers, preferably about 1 to about 5 micrometers, more preferably about 2.0 to about 4.0 micrometers MMAD. The inspiratory flow of the subject, the flow of dispersing gas, or both, then transports the aerosolized dry powder formulation out of the device. Having the container also function as a turbulence chamber has the advantages of enabling a smaller device design, minimizing device cost, and also eliminates the need for the channel between the container and the turbulence chamber.

Additionally, a device for aerosolizing a formulation (e.g., a dry formulation) may include one or more additional containers, for instance, an additional container located in between a first container that contains a pre-filled formulation (e.g., a dry powder formulation) and a turbulence chamber. In one embodiment, the additional chamber may contain a liquid to solubulize or suspend the dry formulation, which liquid is preferably pre-filled and may also contain active pharmaceutical components. When the actuator is actuated, the dry powder drug formulation is forced from the first container, through a first channel that opens to a second container and then through a second channel that opens into a mixing chamber. The energy provided by the actuator is sufficient to cause mixing of the dry powder formulation with the liquid solution in the mixing chamber. The energy supplied by the actuator is also sufficient to cause the mixed drug solution or suspension to be aerosolized, for example by passing it through an exit pore, or an array of pores, of the turbulence chamber thereby generating particles. In the embodiment where the device is an inhaler for pulmonary or nasal administration, the coordinated inspiratory flow of the patient may draw the aerosolized mixed solution from the device via an airway and into the respiratory tract of the patient.

Whether the formulation is liquid or dry, the current invention has the advantage that the large amount of energy required to create the very large surface area of the aerosol need not be supplied by the patient. In some embodiments, the current invention also has the advantage that the device supplies gas flow to dilute and entrain the aerosol, and can be used for non-pulmonary delivery, such as buccal, nasal, ocular, dermal, rectal, or vaginal delivery, where inhalation flow is not available.

An airflow controller and an automatic trigger may also be added to the invention. The automatic trigger may be designed to actuate when the patient is readied for delivery, for example inhaling, or contacting the device with the target organ or region. The airflow controller may be configured to control the patients inhalation rate, or to control the rate at which gas is released from the actuator.

The actuator includes a source of stored potential energy, which supplies the energy required for aerosolization of formulation contained within a container of the device. In certain embodiments, the power source is stored within the actuation chamber and operatively interacts with a piston, such that when the actuator is actuated the power source moves the piston toward the formulation container, preferably also moving a movable wall in the formulation container, and pressurizing the formulation. Alternatively, one face of the piston may function as a wall of the container. In certain embodiments, the piston, with a sealing mechanism, for example an o-ring, forms an air tight seal in a pressurized gas reservoir, and the gas exerts a pressure on the piston, even during storage.

A latch is configured for preventing the deployment of the piston prior to actuation of the actuator. A triggering mechanism is configured for operating the latch. In certain embodiments, a force is applied by depressing the triggering mechanism. In another embodiment, the trigger is actuated by a predetermined minimal inhalation effort achieved by user inhaling through the device. In the preferred embodiment, the triggering mechanism is designed to not be capable of being reset, and can therefore only be operated once, thereby preventing subsequent actuations. In some embodiments, the triggering mechanism is configured to further provide for regulation of fluid flow rates through the device.

In certain embodiments, the piston is held in place by an engaged latch of the actuator and when the triggering means is actuated the latch is disengaged and the piston is free to move in response to the force being exerted by the potential energy power source. In certain embodiments, the piston is operatively connected to a second moveable piston which in turn is in operative communication with the container. In certain embodiments, the triggering of the latch releases the piston, which moves from a first position to a second position, under the force of the power source, which in turn causes the second moveable piston to move from a first to a second position which in turn interacts with the drug container in a manner sufficient to cause the aerosolization of a contained formulation. For instance, in certain embodiments, movement of the piston from a first to a second position applies sufficient force on the container such that a contained formulation is forced through one or more pores of the container and is aerosolized.

The size and mass of the inhaler will depend on the materials used for making the device and the quantity of liquid drug. Typically, thin-walled construction is employed where possible using lightweight aluminum for the actuator and polymers for the remainder of the components such that an inhaler, capable of aerosolizing 50 microliters, measures approximately 9.6 cm in height by 9.3 cm in depth by 3.2 cm in diameter with a mass of about 47 g and includes the liquid formulation.

Representative embodiments of the subject invention are herein set forth below with reference to the included figures.

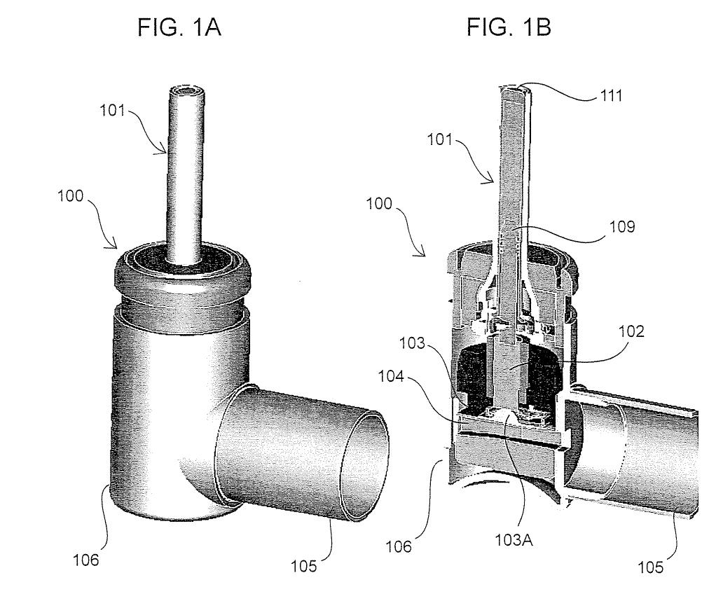

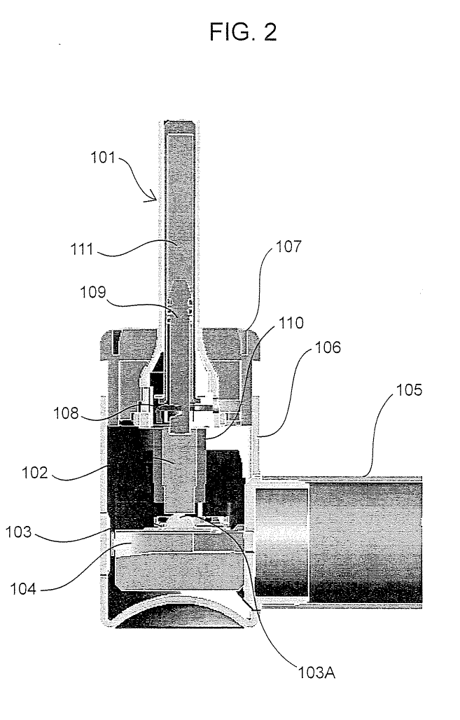

With reference to FIGS. 1A, 1B and 2 a representative delivery device of the invention configured as an inhaler is provided. The device 100 contains a housing 106, a container 103 in the form of a delivery strip, and an actuator 101. The actuator 101 includes a piston 109 and an actuation chamber 111. The piston 109 is moveably associated with and encased within the actuation chamber 111. The actuation chamber 111 contains a power source, such as a stored potential energy source in the form of a compressed gas or spring. The actuator 101 further associates with a trigger 107 and a latch 108. The latch 108 fits within a groove (not shown) within the piston 109. The trigger 107 is operatively connected with the latch 108 such that manipulation of the trigger 107 disengages the latch 108 from its association with the groove of the piston 109. The actuator 101 additionally incorporates a second piston 102, within a sleeve 110, which is aligned with a blister 103A of the delivery strip 103. The delivery strip 103 contains a formulation, such as a liquid drug composition to be delivered to a subject (e.g., a user of the device).

The actuator 101, with incorporated second piston 102, and the delivery strip 103 are clamped to an airway 104 which in turn is attached to the housing 106. A mouthpiece 105 is also attached to the housing 106 in a position to enable a user to inhale an aerosolized dose of the contained formulation. The housing 106 further incorporates the trigger 107 which communicates with the actuator 101 such that a subject who uses the device (e.g., inhaler) may engage the trigger 107 in a downward motion that in turn slides the latch 108 out of the groove of the piston 109 thereby actuating the actuator 101.

Upon actuation, the power source, e.g., a compressed gas contained in the actuation chamber 111 of the actuator 101, exerts sufficient force on the piston 109 so as to cause the piston 109 to drive the second piston 102 into engagement with the blister 103A of the delivery strip 103. The engagement of the second piston 102 with the delivery strip 103 is such that one or more surfaces of the blister of the delivery strip 103 collapse and thereby force the contained formulation through pores contained within the delivery strip 103. The extruded formulation then becomes aerosolized into the airway 104. As the subject inhales during this process, the aerosolized drug formulation travels through the airway 104, continues through the mouthpiece 105, and then enters the subject's respiratory tract. In one embodiment, a safety mechanism, in the form of a removable tab or barrier, is positioned between the trigger 107 and the delivery strip 103 and airway 104 that disables the devices ability to trigger until it is removed.

A damping medium may also be included so as to soften the impact of the piston 102 against the delivery strip 103 such that the force of the piston 102 does not rupture the pores, container, or other features of the delivery strip 103 and the extrusion of the formulation through the pores continues in a smooth fashion rather than in a short burst. Typically the damping medium is contained between the second piston 102 and the sleeve 110. The damping medium may be any fluid capable of damping the interaction of the piston 102 with the delivery strip 103, for instance, the damping medium may be a silica thickened, viscous, synthetic hydrocarbon lubricant grease that has an apparent viscosity of approximately 15,800 poises (Nye Nyogel® 767A). Other damping mediums that may be used include air and various varieties of oil.

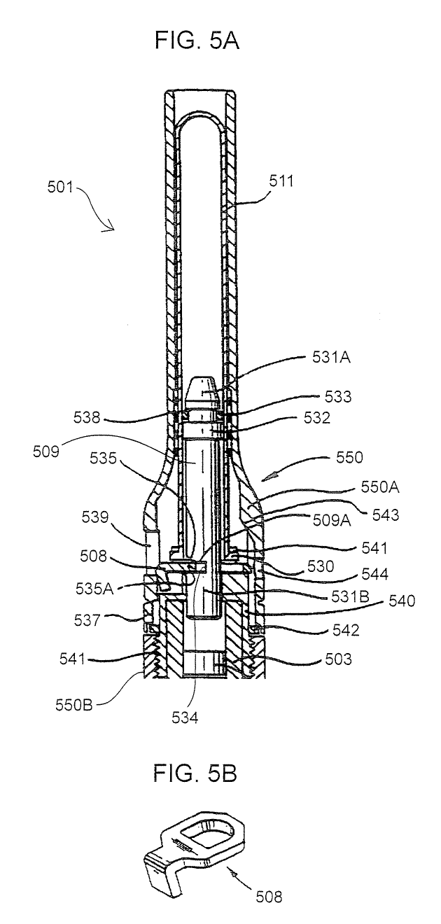

With respect to FIG. 5A, one possible actuator 501 of an embodiment of the invention is provided in greater detail. The actuator 501 includes an actuation chamber 511 in the form of a cylinder, which is closed at its upper end and which contains a power or energy source, for instance a compressed mechanical spring or a gas, typically air or compressed nitrogen, under a pressure which is typically in the range of about 10 psi to about 10,000 psi, preferably about 100 to about 2,000 psi, more preferably about 200 to about 1,000 psi. The energy required for the aerosolization, as characterized in FIG. 4, is provided by the compressed gas spring. The cylinder 511 contains an outer casing or sleeve 550 and houses a piston 509. The piston 509 has a proximal and a distal portion (531A and 531B respectively). The proximal end of the piston 509 has a frustoconical portion 531A and a flange 532 between which is situated an O-ring seal 533. Although FIG. 5 shows only a single o-ring, it is preferable to have two o-rings to ensure that the gas pressure in cylinder is maintained during storage.

Prior to use, the piston 509 is held in the illustrated position by latch 508 which engages the piston 509 in a groove 534 in the piston 509, the upper surface of the groove may form a cam surface 535. The cam surface has a slope of about 2 to about 30 degrees, preferably about 5 to about 15 degrees, more preferably about 6 to about 10 degrees. The latch 508 is shown on a larger scale in FIG. 5B. In the position shown in FIG. 5A the latch is unable to move in a perpendicular direction in relation to the piston 509, because it bears against the inner wall of a sleeve 550.

When the embodiment of FIG. 5A is to be operated, the user removes the safety 537, grasps the upper part of the sleeve 550, and urges the upper sleeve portion 550A downwardly, with respect to the lower sleeve portion 550B. This brings aperture 539 in the wall of the upper sleeve portion 550A into alignment with the latch 508, which is thus able to move sideways from its first position into a second position into the aperture under the influence of the force of the gas within the cylinder 511 acting on the latch via the cam surface 535 formed in the piston 509.

Where the power source is a compressed gas, the pressure within the cylinder 511 may be achieved by filling the cylinder 511 with about 10 to about 100 mgs of gas, or about 20 to about 60 mgs of gas, including about 30 to about 50 mgs of gas. Many different gasses or gas mixtures can be used, including but not limited to air, nitrogen, helium, argon, CO2, and the like. Additionally, the gas may be compressed to the extent that it becomes a liquid. Liquidfied gasses that can be used include but are not limited to CO2, nitrous oxide, chloro-flouro carbons (CFCs), Hydro-flouro alkanes (HFAs) and the like.

The lower end of the cylinder 511 has an outwardly directed flange 530, which enables the cylinder to be held by crimping the flange 530 beneath an outwardly directed flange 541 at the upper end of a coupling 540. The sleeve 550 is formed of an upper sleeve portion 550A within which the cylinder is situated, and a lower sleeve portion 550B. The sleeve portion 550B is connected to the coupling by the interengaging screw threads 541 formed on the inner and outer walls of the sleeve portion 550B and coupling 540 respectively.

In a gas spring powered actuator of the type described above, the gas spring continuously exerts a force on a dispensing member, prior to use, and restraining means are provided for preventing the dispensing member moving under the force of the spring. The actuator is fired by, in effect, moving the actuator into a condition in which the restraining means no longer have a restraining effect, thus permitting the dispensing member to move.

There is, however, a potential problem with transporting and/or preparing such devices for use, in that if the device is to be easily operable by the user, it may be easy, or at least possible, for the device to be accidentally fired during transportation or in preparation prior to use. This is not only wasteful, but also poses a safety hazard to the user. It will be appreciated that it is important that inhalers, or indeed any drug delivery devices with power stored in them, should not be able to trigger prematurely. Similarly, there is a related problem that during assembly, there is a stage wherein the latch 508 is in place restraining the piston 509, but the upper sleeve 550A is not yet in place, and thus cannot restrain the movement of latch 508. The device includes one or more safety mechanisms that operate before the device has been completely assembled to effectively prevent movement of the latch means into the second position where the piston is not constrained, thereby preventing premature firing.

In one embodiment of the invention, described in more detail below, a safety mechanism is incorporated between the trigger and the housing to prevent accidentally movement of the trigger before actuation. Additionally, another safety mechanism may be incorporated into the latch member, which then has a safety position, in which it cannot be moved to its second position by the trigger means, and a non-safety position, in which it can be so moved.

As a precaution against accidental firing, the lower part of the trigger may contain a safety mechanism in the form of a tear-off band or removable block. The lower edge of the first safety mechanism bears against the housing and is bonded to the exterior surface of the housing or is formed integrally therewith. The function of the safety mechanism is to prevent downward movement of the trigger relative to the housing for as long as the safety mechanism is present. The safety mechanism on the trigger need not extend completely around the periphery of the housing or the trigger. Preferably, the safety mechanism is removed by the user immediately prior to triggering the device.

As described above, the trigger of the assembled device is prevented from actuating the device by the presence of the safety mechanism, since until it is removed the device cannot fire. There is, however, a potential problem with assembling such devices prior to use, in that if the device is to be easily operable by the user, it may be easy, or at least possible, for the device to be accidentally fired during the process of manufacture. For instance, during assembly of the device, the penultimate component to be assembled is the housing, which carries the tear-off band (described above). However, before the housing is in place accidental firing is still possible. Accidental firing during the assembly process is a real possibility for several reasons.

First, immediately prior to installation of the housing, there may be a stage in which the partially assembled device has a period of quarantine to check for gas leaks. Secondly, during installation of the housing, the device will be subjected to numerous forces and vibrations arising from the assembly equipment. Even after installation of the housing, the assembly stresses arising as the device is handled during the final steps of the manufacturing process may be sufficient to cause accidental firing, despite the presence of the tear-off band.

Certain embodiments of the present invention provide means for overcoming this problem. For instance, to deal with this problem the device may have an additional safety mechanism. Referring again to FIG. 5, an additional safety mechanism is provided by forming the slot in the piston not only with the cam surface 535 but also with a locking surface 535A which extends perpendicular to the axis of the piston and is located radially inwardly of the cam surface 535. To enable the combination of cam surface 535 and locking surface 535A to be used in the intended manner, the upper sleeve portion 550A is provided with an opening 144 that extends there through at a location that, prior to the device being fired is aligned with the end of the latch 508 remote from the slot in the piston.

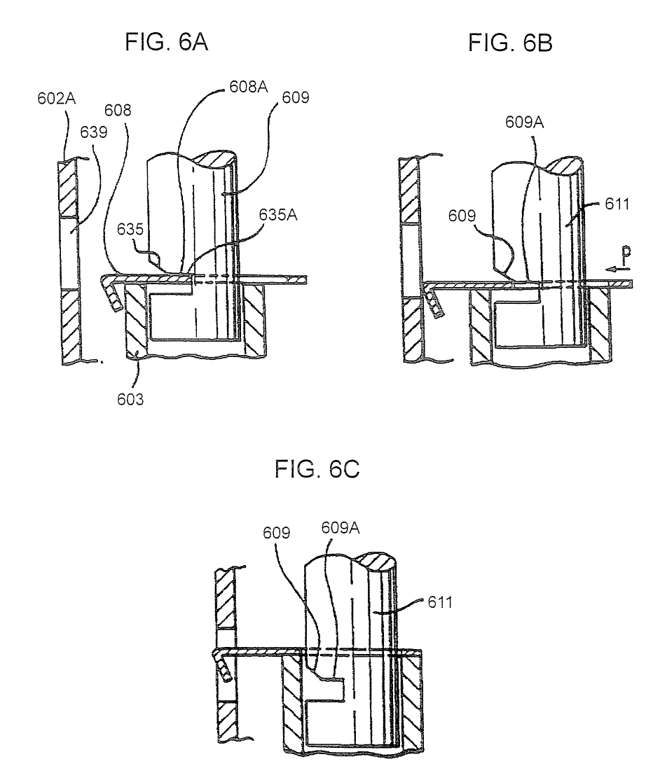

The safety mechanism may be seen in greater detail with reference to FIG. 6. When the latch 608 and piston 609 are initially assembled with one another, the latch 608 occupies the position shown in FIG. 6A, which is a safety position. Here, the piston-engaging latch portion 608A is acted on by the locking system 635A. Friction forces ensure that the latch remains engaged with the locking surface; typically the piston exerts a force of at least 10N, so the latch is held in a vice-like grip.

Once the device has been assembled, preferably completely, and at least to the extent of the upper sleeve portion 550A being in place, it is cocked by inserting a tool through opening 544 to push the latch in the direction of the arrow P in FIG. 6A into the position shown in FIG. 6B (See also FIG. 5). In this position the piston-engaging latch portion 508A is in contact with the radially inner end of the cam surface 635. When the device is actuated as described above it is able to fire because the latch moves to the position shown in FIG. 6C. The user can cock the device prior to the removal of the trigger safety mechanism, or preferably a mechanism is provided that combines the action of removing the trigger safety mechanism and cocking the device. In one embodiment, the device is cocked in the factory after the assembly of the actuator.

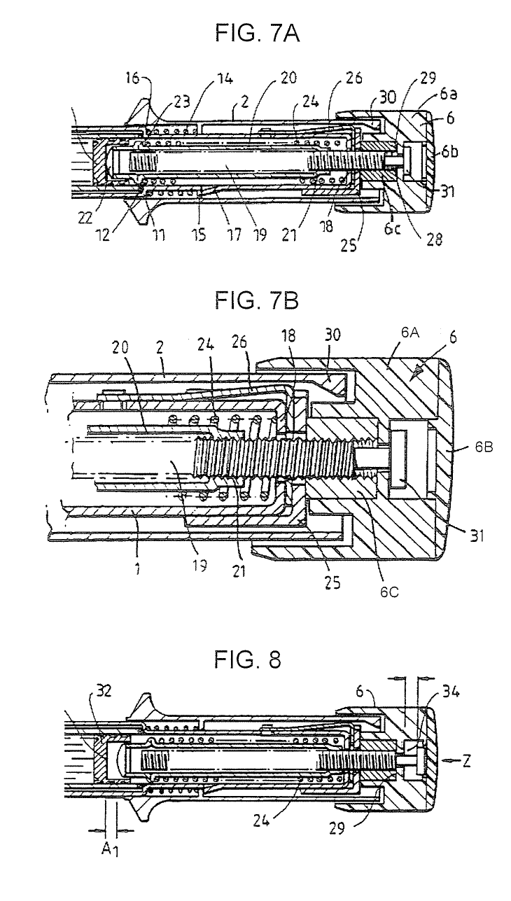

In another representative embodiment of the subject invention, a mechanical compression spring is provided as the energy source of the actuator. With reference to FIGS. 7 through 11, a spring actuated delivery device of the invention is provided.

In FIGS. 7A and 7B, the actuator is shown with a free piston 32. The sliding sleeve 2 is assembled co-axially on body 1 and is urged away rearward by a spring 14 supported by a shoulder 16 on body 1 and acting on a shoulder 15. The extent of the rearward movement is limited by shoulder 15 resting on one or more stops 17. A cam 30 is formed inside the sleeve, so that when the sleeve is moved forward, the cam strikes a latch 26 to initiate the actuation.

Support flange 18 is formed on the end of the body 1 and has a hole co-axially therein through which passes a threaded rod 19, which may be hollow to save weight. A tubular member 20 is located coaxially within the rear portion of the body 1 and has an internal thread 21 at one end into which the rod 19 is screwed. The other end of the tubular member 20 has a button having a convex face 22 pressed therein. Alternatively, the tubular member 20 may be formed to provide a convex face 22. A flange 23 is formed on the tubular member, and serves to support a spring 24, the other end of which abuts the inside face of support flange 18. In the position shown, the spring 24 is in full compression, and held thus by the nut 6 which is screwed onto threaded rod 19, and rests against the face of the bridge 25. In the illustrated embodiment the nut 6 consists of three components, held fast with one another, namely a body 6A, an end cap 6B and a threaded insert 6C. The insert 6C is the component that is screwed on to the rod 19, and is preferably made of metal, for example brass. The other components of the nut can be of plastics materials.

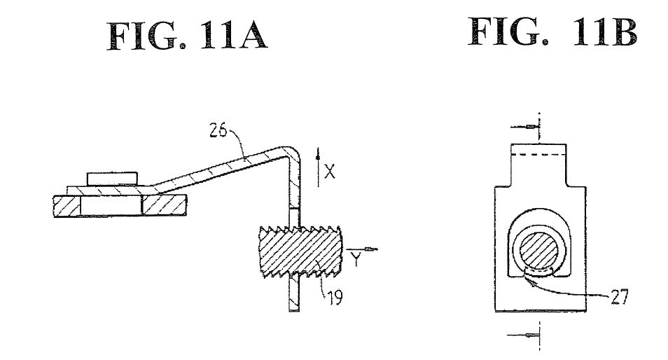

Beneath the bridge and guided by the same is a latch 26 which is attached to the body 1 and resiliently engaged with one or more threads on the screwed rod 19. The latch 26 is shown in more detail in FIG. 11, and is made from a spring material and has a projection 27 that has a partial thread form thereon, so that it engages fully with the thread formed on rod 19. The latch 26 is attached to body 1 and has a resilient bias in the direction of arrow X, thus maintaining its engagement with the thread on rod 19. Movement against the direction of arrow X disengages the latch from the thread. As will be described, the rod 19 will be translated without rotation in the direction of arrow Y when setting the impact gap, and the latch 26 will act as a ratchet pawl. The thread on rod 19 is preferably of a buttress form (each thread has one face which is perpendicular or substantially perpendicular, for instance, at about 5°, to the axis of the rod, and the other face is at a much shallower angle, for instance, at about 45°), giving maximum strength as a latch member, and a light action as a ratchet member.

Referring again to FIG. 7A, nut 6 is screwed part way onto threaded rod 19, so that there is a portion of free thread 28 remaining in the nut 6, defined by the end of rod 19 and stop face 29 in nut 6. A stop pin 31 has a head which bears against the stop face 29, and a shaft which is fixedly secured to the inside of rod 19, for example by adhesive. The stop pin 31 prevents the nut 6 being completely unscrewed from rod 19, since when the nut 6 is rotated anticlockwise, it will unscrew from the rod 19 only until the head of pin 31 contacts the face of the recess in the nut 6 in which it is located. The pin 31 also defines the maximum length of free thread in nut 6 when fully unscrewed.

Referring to FIG. 8, the first stage in the operating cycle is to rotate the nut 6 on threaded rod 19 in a clockwise direction (assuming right-hand threads, and viewing in direction arrow Z). The rod 19 is prevented from turning, since the friction between the screw thread and the latch 26 is much higher than that between the nut 6 and the rod 19. This may be because the nut is unloaded, whereas the rod 19 has the fall spring load engaging it with the latch 26. The rod 19 moves into the nut 6 as far as the stop face 29. Alternative ways could be used to prevent the rod 19 from turning, for example, using a ratchet or the like, or a manually operated detent pin. Since the threaded rod is attached to the tubular member 20, by the interengagement of the thread on rod 19 with the thread 21 on member 20, the latter is also moved rearwards (i.e., to the right as viewed in FIG. 2), increasing the compression on spring 24, and thus creates a gap A₁ between the convex face 22 of the tubular member 20 and the inner face 33 of piston 32. When the rod 19 is fully screwed into nut 6 the stop pin 31 projects a distance A₂ from face 34 that is equal to the gap A₁.

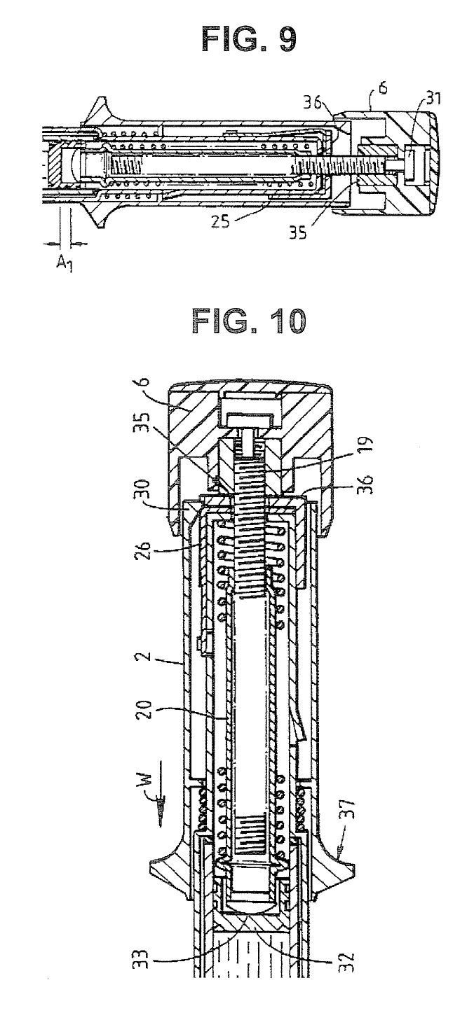

Referring to FIG. 9, nut 6 is now rotated anticlockwise until it contacts stop pin 31, which locks the nut 6 to the threaded rod 19. There is now a gap between face 35 on nut 6 and the abutment face 36, which gap is equal to gap A₁. Continued rotation of the nut now rotates the threaded rod also, because of the attachment of the shaft of the pin 31 to the side of the rod 19, and unscrews it in a rearward direction. The face 35 on nut 6 thus moves further away from its abutment face 36 on bridge 25. The increase in the gap is equivalent to the required stroke of the piston, and thus the total gap is the sum of the impact gap A₁ and the required stroke. The nut 6 has markings on the perimeter which are set to a scale on the sliding sleeve 2, in the manner of a micrometer. The zero stroke indication refers to the position of nut 6 when it first locks to the threaded rod 19, and immediately before the threaded rod is rotated to set the stroke.

Referring to FIG. 10, the actuator is now ready to actuate. Force is applied on the trigger 37 in the direction of arrow W. The sliding sleeve 2 compresses spring 15 and moves forward so that the force is transmitted through spring 14 to the body 1. When the contact force has reached the predetermined level, the cam 30 on sliding sleeve 2 contacts latch 26 and disengages it from threaded rod 19. The spring 25 accelerates the tubular member 20 towards the piston through the distance A₁, and the convex face 22 strikes the face 33 of piston 32 with a considerable impact. The tubular member 20 thus acts as an impact member or piston. Thereafter the spring 24 continues to move the piston 32 forward until the face 35 on nut 6 meets the face 36 on bridge 25. The impact on the piston causes within the formulation of the delivery strip (for instance, an AERx strip) a very rapid pressure rise—effectively a shock wave—that appears almost simultaneously at the delivery strip. The follow-through discharge of the formulation is at a pressure that is relatively low but sufficient to keep extruding the formulation from the strip.

Spring 24 should be given sufficient pre-compression to ensure reliable actuations throughout the full stroke of the piston. A 30% fall in force as the spring expands has been found to give reliable results. Alternatively, a series stack of Belleville spring washers in place of a conventional helical coil spring can give substantially constant force, although the mass and cost will be slightly higher.

In accordance with this embodiment, the power source of the actuator is a spring which is pre-loaded by the manufacturer. Thus the user merely rotates the single adjustment nut and then presses the trigger of the actuator. The force to move the piston is provided by the spring, (as described, a compression spring) which is initially in its high energy state (i.e., compressed in the case of a compression spring). The piston member is moved by permitting the spring to move to a lower energy state (i.e., uncompressed, or less compressed, in the case of a compression spring).

Many variations in the described embodiments are possible. For example damping grease may be retained within a circumferential groove on the body that is a close sliding fit within the operating sleeve. It is simple to vary the viscosity or running clearance to obtain the desired damping characteristics. Further modifications to the damping characteristics are possible by using dilatant or shear-thickening compounds. However, in practice, the range of forces applied by users is within sensible limits. While grease has been discussed as a damping medium, similar results may be obtained by using air or oil damping devices—usually a cylinder and piston combination, i.e. a so-called “dashpot”, wherein a fluid substance is caused to flow through a restriction, thereby to resist motion. Other viscous damping devices employ a vane, or a plurality of vanes, spinning in a damping medium, for example air, and these may be used if appropriate to the particular application.

Many medical conditions can be treated using the invention. In a preferred embodiment, the system is used to treat conditions that are acute and don't need daily dosing. For acute conditions that occur relatively rarely but when they occur have very negative effects including but not limited to pain, imminent loss of life, permanent injury, strong discomfort, or loss of work time, a portable, small, easy to use system such as the present invention can be used. These conditions include but are not limited to pain, migraine, acute injury, nausea, poisoning, leg cramps, depression, anxiety, panic attacks, vertigo, sleep disorders, paranoia, myocardial infarction, stroke, seizure, and shock (including anaphylactic shock and the like). Additionally, the inhaler may be carried by military personnel and civilians as a countermeasure against exposure to bio-terror agents including but not limited to nerve gas, ricin, anthrax, botulism, and small pox. Additionally, medical conditions where rapid onset is preferred, and for other reasons the user wants a very simple to operate device that does not require dosage form loading or complex device manipulations or cleaning, the devices of the invention are useful. Such conditions may include conditions related to sexual dysfunction.

The formulation is typically a flowable composition such as a liquid or dry powder. In certain embodiments the formulation is a solution of or suspension in water, ethanol, or a combination thereof. The liquid formulations of the invention may include preservatives or bacteriostatic type compounds. Typically, the formulation includes an active agent, for instance, a pharmaceutically active drug which may further include a pharmaceutically acceptable carrier. The formulation may include the active agent without a carrier if the active agent is freely flowable and can be aerosolized.

A wide variety of liquid and dry powder drugs and formulations thereof may be delivered to subjects via the pulmonary route using a device of the invention. Some examples include, but are not limited to, the following: PDE5 inhibitors such as tadalafil or vardenafil may be delivered with the inhaler to treat erectile dysfunction; epinephrine, as a bronchodilator for asthma; atropine, as an antidote for nerve gas poisoning; fluoroquinolones, such as ciprofloxacin used against anthrax; benzodiazepines, for the treatment of anxiety or insomnia; methocarbamol, as a muscle relaxant for leg cramps; and ipratropium bromide, for the treatment of obstructive lung diseases.

Other examples of active agents that can be delivered as liquid formulations may further include the pharmaceutically active drug being contained in a formulation-based drug delivery platform that comprises liposomes, micelles, polymers, dendrimers, nanotubes, buckyballs, microporous structures, nanoporous structures, and layer-by-layer colloidal systems. For example, ciprofloxacin may be combined with liposomes in the formulation in order to achieve a long lasting dose. Likewise, several of the examples that can be delivered as dry powder formulations may further comprise the pharmaceutically active drug being contained in a formulation-based drug delivery platform that comprises polymers, microporous structures, and nanoporous structures. An example here includes PDE5 inhibitors that may be combined with a polymer to achieve a long lasting dose.

Drug formulations for use in the device may be made into dry powders using methods well known and commonly practiced in the pharmaceutical industry to create dry powders of drugs, and include but are not limited to lyophilization, milling, spray drying, and precipitation, including precipitation by co-solvents, especially gas co-solvents. These processes are known to have the capability of creating particles of approximately 1 to 3 micrometers in physical diameter that are needed for the dry powder inhalers of the present invention. Lyophilized drug formulations are also packaged into the containers of the inhalers using methods that are also well known and commonly practiced in the pharmaceutical industry.

In certain embodiments, the formulations are sterilized and placed in individual containers in a sterile environment. Useful formulations may include compositions currently approved for use with nebulizers. However, nebulizer formulations must, in general, be diluted prior to administration. Other formulations may include presently approved parenteral formulations, or novel formulations optimized in terms of drug concentration, surface tension, viscosity, or any other formulation properties to be optimized for use with the present invention. The active agent may be a drug, for instance, a small molecule drug, a nucleic acid, peptide or protein, or any other type of drug. The formulation may contain a single active ingredient, 2 active ingredients, or any number of active ingredients.

A device of the invention may further include a sterile over-wrap to protect said drug delivery system before use by the patient. The over-wrap may be a polymeric bag that is optionally sterilized (e.g. gamma irradiated), placed around the drug delivery system, and then vacuum-sealed for storage. The bag is designed to maintain the stability of the drug delivery system for the shelf life of the drug formulation contained therein with the added feature that the package is highly water and dust resistant. This enables the device to be carried and used in extreme environments such as rainy weather, aquatic environments, purses and pockets, and in the desert.

The embodiments thus described provide inexpensive, compact, convenient and easy-to-use single dose disposable inhalers, capable of aerosolizing a liquid formulation of drug for inhalation by the patient. The power source is preferably a mechanical spring or pressurized gas source that is pre-loaded by the manufacturer, and the formulation container is also preferably pre-filled and assembled into the inhaler.

In one embodiment, wherein the device of the invention in an inhaler for pulmonary administration, the usage would be as follows: the user first removes the outer over-wrap, if included. If a mouthpiece is not already attached the user removes the mouthpiece from a storage location (e.g., on the housing) and places the mouthpiece onto the mouthpiece location of the housing. The user then places his or her mouth on the mouthpiece, making a seal with their lips. The user then sets any safety mechanisms in the ready to deliver state (e.g., removes any tabs or blocks that prevent activation of the trigger), and begins inhalation while actuating the actuator (e.g., depressing the trigger). In one embodiment, the device is breath actuated, i.e. the act of beginning inhalation itself triggers actuation of the device. Following actuation, the device is disposed of.

Accordingly, the devices of the invention are useful in methods for treating a condition, for instance, erectile dysfunction, asthma, nerve gas poisoning, anxiety, insomnia, cramps, or an obstructive lung disease. The methods may include one or more of the following steps. First, an intrapulmonary drug delivery device, such as those described above, is obtained. Accordingly, the device may include one or more of the following components: an actuator, configured for actuating said device; a store of potential energy, configured for aerosolizing a contained flowable formulation when said device is actuated; a container containing said flowable formulation; a mouthpiece, configured for allowing the passage of an aerosolized formulation to a user of the device; a safety mechanism, configured for preventing the unintended actuation of the device; and a housing, configured for interconnecting the components of the device. Once a suitable device has been obtained (for instance a first device) the device is positioned for use (e.g., a user places the mouth of the user at a user interface, such as over a mouthpiece), any safety mechanisms included are disengaged, and the device is actuated so as to aerosolize said contained flowable formulation and produce an aerosolized formulation. The aerosolized formulation is then inhaled by the user. After use the device is then disposed of and at some later time a new (e.g., a second) intrapulmonary drug delivery device is obtained and used in accordance with the steps provided above.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

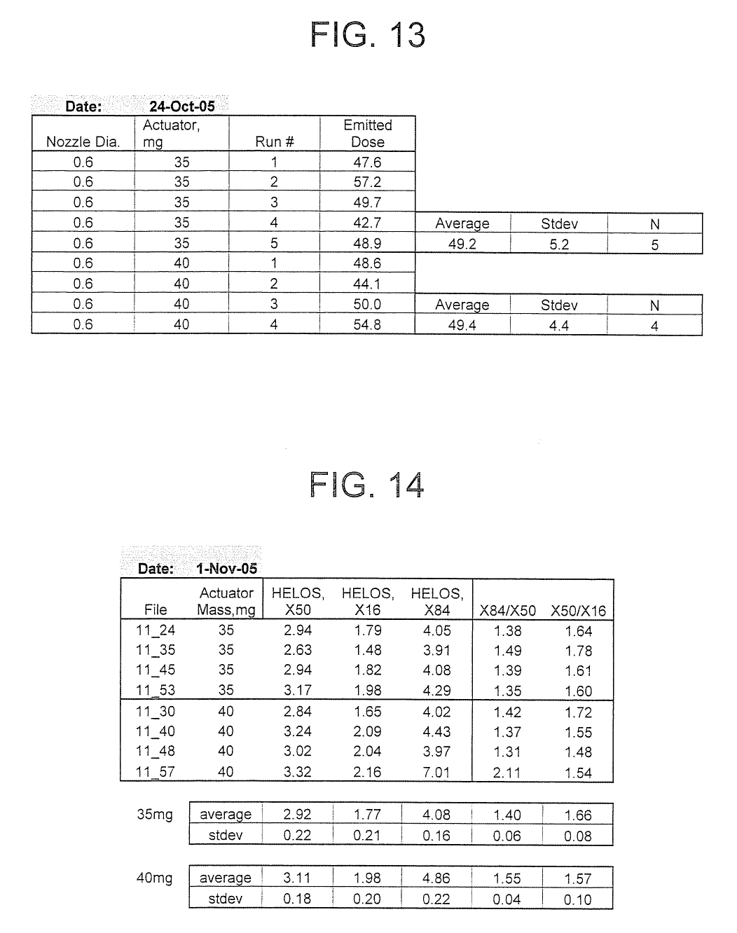

The embodiment of the invention as shown in FIGS. 1, 2, 5 and 12 was used to quantify the efficiency of aerosol generation. The device was used with 0.6 micrometer nozzles and the container was filled with 50 μL of an aqueous solution of 30 mg/mL sodium cromoglycate. The actuator was charged with gas masses of 35 mg and 40 mg. As shown in FIG. 13, a gas mass of 35 mg provided an average ED of 49.2% with a standard deviation of 5.2 (N=5) while a gas mass of 40 mg provided a similar average ED of 49.4% with a standard deviation of 4.4 (N=4). As shown in FIG. 14, the particle sizes (MMAD) using a gas mass of 35 mg was 2.92 micrometers with a standard deviation of 0.22, while a gas mass of 40 mg generated median particle sizes of 3.11 micrometers with a standard deviation of 0.18.

### Example 2

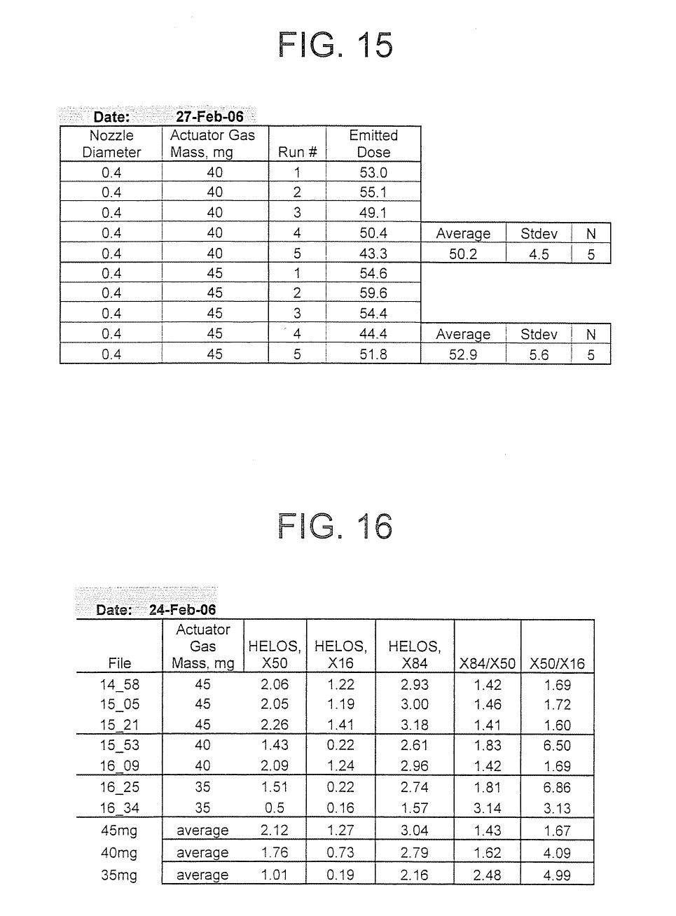

FIG. 15 is a table of ED data using a version of the inhaler that is similar to that used in example 1, except that 0.4 micrometer nozzles were used, and the actuator were charged with gas masses of 40 and 45 mg. Emitted doses were measured that respectively averaged 50.2% and 52.9% with standard deviations of 4.5 and 5.6 (both were N=5).

FIG. 16 presents particle size distribution data, again using the inhaler of example 1, with 0.4 micrometer nozzles and 30 mg/mL sodium cromoglycate at actuator gas masses of 45 mg, 40 mg and 35 mg. Using a gas mass of 45 mg, an average particle size of 2.12 micrometers was measured, and the delivery time was found to be in 2.67 seconds (N=3). A gas mass of 40 mg was found to have an average particle size of 1.76 micrometers with a delivery time of 2.70 seconds (N=2), and a gas mass of 35 mg was found to have an average particle size of 1.01 micrometers and a delivery time of 3.20 seconds (N=2).

The instant invention is shown and described herein in a manner which is considered to be the most practical and preferred embodiments. It is recognized, however, that departures may be made there from which are within the scope of the invention and that obvious modifications will occur to one skilled in the art upon reading this disclosure.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

That which is claimed is:

1. A single dose drug delivery device, comprising:
a source of stored energy;
a triggering mechanism;
a container forming part of the device and holding a flowable formulation consisting of only a single dose of pharmaceutically active drug;
a mechanism for transferring said stored energy to the container.

2. The drug delivery device of claim 1, wherein the energy source is chosen from: a mechanical spring; a pressurized gas; and a chemical composition capable of releasing energy in a chemical reaction.

3. The drug delivery device of claim 1, wherein the drug delivery device is an inhaler and the drug is selected from the group consisting of sildenafil, tadalafil, vardenafil.

4. The drug delivery device of claim 2, wherein the flowable formulation is a formulation chosen from a liquid and a dry powder; and
wherein the mechanism for transferring comprises a cylinder and a piston slidably positioned in the cylinder.

5. The drug delivery device of claim 4, wherein said device further comprises:
a safety mechanism having a locked position and a ready position where the safety must be set in the ready position to actuate the trigger to release the stored energy.

6. The inhaler of claim 5, wherein the source of stored energy is a mechanical spring and the device further comprises a mouth piece.

7. The drug delivery device of claim 4, wherein the formulation is a dry powder, and the device further comprises:
a turbulence chamber and a connecting channel, wherein the connecting channel connects said container to the turbulence chamber.

8. The drug delivery device of claim 7, further comprising:
an additional chamber, wherein said additional chamber further comprises a liquid solution configured for mixing with said dry powder formulation.

9. The drug delivery device of claim 1, wherein the drug is chosen from: sildenafil, tadalafil, vardenafil, epinephrine, ipratropium bromide, methocarbamol, benzodiazepine, atropine, and liposomal ciprofloxacin.

10. The drug delivery device of claim 4, wherein said flowable formulation comprises a controlled release component chosen from: liposomes, micelles, polymers, dendrimers, nanotubes, buckyballs, microporous structures, nanoporous structures, layer-by-layer colloidal systems.

11. The drug delivery device of claim 4, further comprising:
a sterile overwrap encasing the entire device.

12. The drug delivery device of claim 4, wherein the energy source comprises a chamber of compressed gas.

13. The drug delivery device of claim 12, wherein the compressed gas has a pressure in the range of about 10 psi to about 10,000 psi.

14. The drug delivery device of claim 12, wherein the compressed gas has a pressure in the range of about 100 psi to about 2,000 psi.

15. The drug delivery device of claim 12, wherein the compressed gas has a pressure in the range of about 200 psi to about 1,000 psi.

16. The drug delivery device of claim 4, wherein the device is an inhaler, the formulation is a liquid formulation and the device further comprises a nozzle.

17. The drug delivery device of claim 5, wherein said safety mechanism comprises a latch configured in a manner such that it can engage and disengage from a receiving indentation in the piston.

18. The drug delivery device of claim 17, wherein when said latch is in an engaged position the latch moveably associates with the indentation in said piston and thereby prevents movement of the piston, the trigger mechanism is configured for moving said latch out of the indentation.

19. The drug delivery device of claim 18, wherein said device is an inhaler and the trigger is activated by user inhalation.

20. The drug delivery device of claim 18, wherein said trigger is capable of being depressed and when depressed disengages from the indentation allowing said piston to move from a first position to a second position.

21. The drug delivery device of claim 19, wherein said trigger further comprises an automatically actuated mechanism for actuating the actuator component as a result of mechanical resistance on the inspiratory flow of a patient causing mechanical communication with said automatically actuated mechanism for the actuator component.

22. The drug delivery device of claim 20, wherein said piston is operatively connected to a second piston in a manner sufficient to move said second piston from a first to a second position when the trigger is actuated wherein when said second piston is moving toward said second position, the piston contacts said container and wherein said container comprises a surface which moves and reduces container size and so that container contents are expelled from the container.

23. The drug delivery device of claim 22, wherein said container comprises a membrane comprising a plurality of pores.

24. The drug delivery device of claim 25, wherein said piston contacts said container with a force sufficient to force formulation through the pores and aerosolize said contained flowable drug formulation.

25. The drug delivery device of claim 4, wherein the flowable formulation is a dry powder, and wherein said potential energy store comprises a pressurized gas, and actuation of the device causes the release of the gas, which flows through said dry powder formulation, causing it to disperse into particles of the dry powder formulation.

26. The drug delivery device of claim 24, further comprising:
a mouth piece configured for allowing a user to inhale said aerosolized drug formulation; and
an airflow rate controller that controls the inspiratory flow rate of a user.

27. An intrapulmonary drug delivery system, comprising a plurality of drug delivery devices, wherein each device comprises:
(a) a single dose container, holding a flowable formulation consisting of only a single dose of a pharmaceutically active drug, the container forming an integral part of the device;
(b) a store of energy, configured for aerosolizing the flowable formulation when the energy is released;
(c) an actuator, configured for releasing the store of energy;
(d) a mouthpiece, configured for allowing the passage of an aerosolized formulation to a user; and
(e) a safety mechanism, configured for preventing the unintended actuation of the actuator.

28. A method of treatment, comprising:
(a) removing an intrapulmonary drug delivery device from packaging, wherein said device comprises the following components:
(1) a container forming an integral part of the device, the container holding flowable formulation consisting of only a single dose of a pharmaceutically active drug;
(2) a store of potential energy, configured for aerosolizing the flowable formulation upon release of the energy;
(3) an actuator, configured for releasing the store of energy;
(4) a mouthpiece, configured for allowing the passage of an aerosolized formulation to a user; and
(5) a safety mechanism, configured for preventing the unintended actuation of the device;
(c) disengaging said safety mechanism;
(d) actuating said actuator so as to release the energy and aerosolize the flowable formulation and produce an aerosolized formulation;
(e) inhaling said aerosolized formulation; and
(f) disposing of said intrapulmonary drug delivery device and its packaging.

29. The method of claim 28, further comprising repeating (a)-(f) a plurality of times with a new device each time.

30. The method of claim 28 wherein said the method is carried out to treat a condition chosen from erectile dysfunction, asthma, nerve gas poisoning, anxiety, insomnia, cramps, and an obstructive lung disease.
